(19) United States
(12) Patent Application Publication
(10) Pub. No.: US 2002/0183628 A1
(43) Pub. Date: Dec. 5, 2002

(54) Pressure sensing endograft

(76) Inventors: Sanford Reich, Providence, RI (US); Edward Theodore Bullister, Weston, MA (US)

Correspondence Address: FRANCIS L CONTE, 6 PURITAN AVENUE, SWAMPSCOTT, MA, 01907

(21) Appl. No.: 10/160,741

(22) Filed: May 31, 2002

Related U.S. Application Data
(60) Provisional application No. 60/296,012, filed on Jun. 5, 2001.

(51) Int. Cl. A61B 5/02

(52) U.S. Cl. 600/486

(57) ABSTRACT

An endovascular implant or endograft includes a tubular sleeve having integral inner and outer layers. A pressure sensor is embedded between the two layers and is covered thereby. And, the sleeve is flexible at the pressure sensor to permit transfer of pressure through the sleeve for detection by the pressure sensor in use.

This application claims the benefit of U.S. Provisional Application No. 60/296,012; filed Jun. 5, 2001.

This invention was made with United States Government support under Cooperative Agreement No. 70NANB7H3059 awarded by NIST. The United States Government has certain rights in the invention.

### BACKGROUND OF THE INVENTION

The present invention relates generally to endovascular implants, and, more specifically, to their construction, use, and monitoring.

A common procedure for the treatment of aneurysms, for example, abdominal aortic aneurysms (AAAs), is the use of endovascular implants or grafts, referred to herein as endografts. In this procedure, a collapsed endograft is guided to the site of the aneurysm with an arterial catheter. The endograft is positioned to span the aneurysm sac and expanded so that the ends of the endograft form a seal with the aorta upstream and downstream of the aneurysm. The arterial pressure is then borne by the endograft, and the pressure within the aneurysm is relieved.

A common complication of this procedure is endoleakage. Endoleakage is leakage around the ends of the endograft. Endoleakage occurs when the ends of the endograft do not completely seal with the aortic wall.

Another common complication is retrograde flow into the aneurysm sac through collateral arteries. Both these conditions can lead to repressurization and possible rupture of the aneurysm sac.

These conditions generally can be detected with CT scans. However, the failure to visualize endoleaks does not preclude their presence. Furthermore, the possibility of endoleaks is open-ended, so that all patients with AAA endografts should be followed for life with CT scans.

The current monitoring procedures using CT scans give limited data at infrequent intervals and at high cost.

Accordingly, an improved endovascular implant is desired for reducing cost and improving use thereof.

### BRIEF SUMMARY OF THE INVENTION

An endovascular implant or endograft includes a tubular sleeve having integral inner and outer layers. A pressure sensor is embedded between the two layers and is covered thereby. And, the sleeve is flexible at the pressure sensor to permit transfer of pressure through the sleeve for detection by the pressure sensor in use.

### BRIEF DESCRIPTION OF THE DRAWINGS

The invention, in accordance with preferred and exemplary embodiments, together with further objects and advantages thereof, is more particularly described in the following detailed description taken in conjunction with the accompanying drawings in which:

FIG. 1 is a schematic view of an endograft implanted in the aorta of a living patient to repair or bridge an aneurysm in accordance with an exemplary embodiment of the present invention.

FIG. 2 is an enlarged partly sectional view of a portion of the endograft illustrated in FIG. 1 showing an exemplary pressure sensor embedded therein.

FIG. 3 is a radial cross sectional view through the middle of the endograft illustrated in FIG. 1 within the aneurysm showing two exemplary pressure sensors embedded therein for measuring external and internal pressure of the endograft.

FIG. 4 is a radial sectional view through one end of the endograft implanted in the aorta illustrated in FIG. 1 showing three exemplary pressure sensors embedded therein.

FIG. 5 is an enlarged sectional view of one of the embedded pressure sensors illustrated in FIG. 4.

FIG. 6 is a schematic view for implanting the endograft illustrated in FIG. 1 using a balloon catheter.

FIG. 7 is a isometric view of an exemplary stent usable with the endograft illustrated in FIG. 1 and modified to include an additional pressure sensor integrated therewith in accordance with another embodiment of the present invention.

FIG. 8 is an enlarged view of a portion of the stent illustrated in FIG. 7 showing the pressure sensor fixedly joined therein in accordance with an exemplary embodiment.

FIG. 9 is an enlarged view of the stent illustrated in FIG. 7 showing a box frame for trapping the pressure sensor in the stent in accordance with another embodiment of the present invention.

FIG. 10 is a flowchart representation of analysis of selective pressures monitored in the endograft illustrated in FIG. 1 for detecting leakage around the endograft during use.

FIG. 11 is an equivalent electrical circuit representative of leakage around the endograft illustrated in FIG. 1.

### DETAILED DESCRIPTION OF THE INVENTION

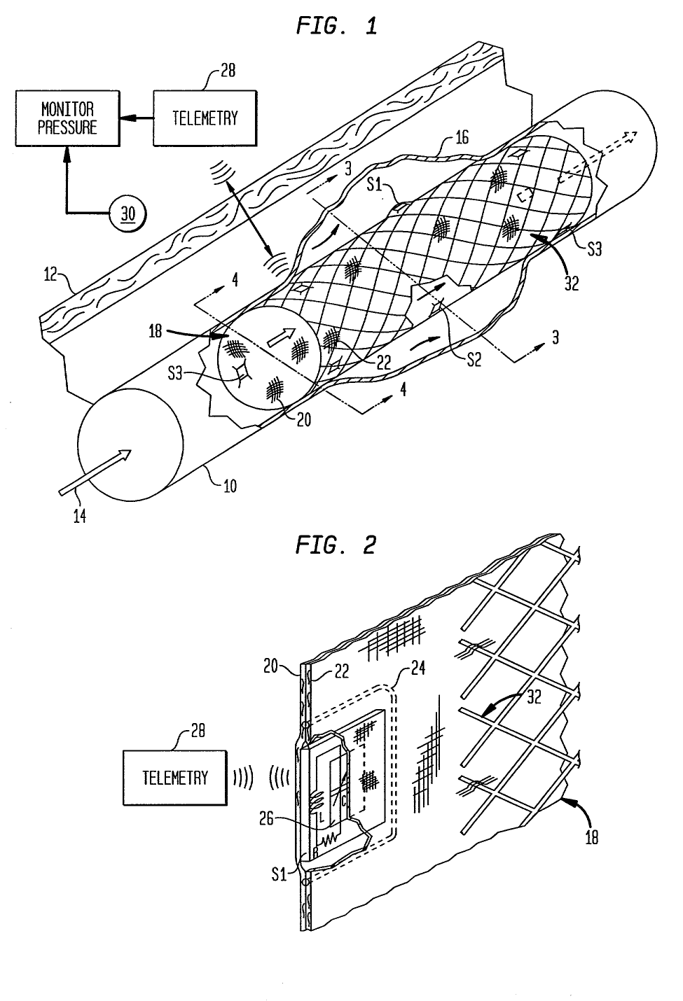

Illustrated schematically in FIG. 1 is an exemplary body vessel or lumen 10 found under the skin 12 inside the body of a living human patient. For example, the vessel 10 may be the aorta of the heart and carries a body fluid or liquid 14 such as blood flow.

In the exemplary embodiment illustrated in FIG. 1 the vessel 10 includes an aneurysm 16 in the form of an enlarged sac in which the normally tubular vessel has a locally enlarged weak portion. The aneurysm is repaired by the introduction of an artificial endovascular implant or endograft 18 implanted inside the vessel to bridge the aneurysm and return this region of the vessel to the normal tubular shape.

FIG. 2 illustrates an enlarged portion of the endograft illustrated in FIG. 1 which is in the preferred form of a two-ply tubular sleeve defined by integral inner and outer plies or layers 20, 22. In the preferred embodiment, the sleeve layers 20, 22 are in the form of a woven fabric of Dacron, for example. Any conventional material may be used for the endograft.

In accordance with the present invention, the endograft is provided with the two plies for integrating therein one or more pressure sensors S1, S2, and S3, for example. Each pressure sensor is self-contained and sufficiently small in size and configuration for being embedded or trapped wholly between the inner and outer layers and completely covered thereby.

In the exemplary embodiment illustrated in FIGS. 1 and 2, the inner and outer layers 20, 22 are coextensive between opposite ends of the sleeve, with each layer being tubular and concentric with the opposite layer. The two layers may be interwoven during manufacture or may be separately produced and joined together by suitable stitching.

As shown in FIG. 2, each of the pressure sensors, such as S2, is embedded between the two plies and may be locally captured therein by surrounding stitching 24. In this way, each pressure sensor is trapped in the endograft itself and cannot be liberated inside the patient during use.

In alternate embodiments, the endograft sleeve could be a single ply woven fabric, for example, with small fabric patches sewn over each of the pressure sensors for embedding them in the endograft. Such fabric patches may be located either inside or outside the endograft sleeve.

As initially illustrated in FIG. 2, the fabric material of the endograft sleeve is preferably flexible at each of the pressure sensors to conform the shape of the sleeve to the pressure sensor and to permit transfer of pressure through the sleeve to the pressure sensor for accurate pressure sensing capability.

The several pressure sensors illustrated in FIG. 1 are preferably identical to each other, and FIG. 2 illustrates an exemplary configuration thereof. Each pressure sensor preferably includes a flat pressure sensing surface or diaphragm 26, and the fabric sleeve is at least locally flexible in the vicinity of each pressure sensor to conform flat with the flat diaphragm. In this way, the endograft sleeve will not obstruct proper operation of the miniature pressure sensors.

Each pressure sensor S1-3 may have any conventional form with its size being preferably a small as possible. For example, a preferred form of a solid-state transducer pressure sensor for use in the endograft 18 is disclosed in an article entitled “A Wireless Batch Sealed Absolute Capacitive Pressure Sensor,” by Akar, et al., as published beginning at page 585 of Eurosensors XIV, the 14th European Conference on solid-state transducers, Aug. 27-30, 2000, Copenhagen, Denmark. Particular advantage of these solid-state transducers is their minute size, telemetric capability, and small silicon diaphragms which form one plate of a capacitor used for accurately measuring pressure thereagainst.

The silicon diaphragm 26 is illustrated in FIG. 2 along with a schematic representation of the variable capacitor C formed thereby in a circuit including a resistor R and an inductor L. A suitable telemetry device 28 includes an electrically powered external coil or inductor in a circuit with another resistor which can be used for inductively coupling each of the pressure sensors with the telemetry device 28 for detecting pressure sensed by the pressure sensor.

In this way, the endograft and its integrated pressure sensors S1-3 may be implanted inside the patient, with the pressure being detected externally of the patient by the remote telemetry device 28 located outside the skin 12. After initial implantation of the endograft, no orifices through the skin or additional surgery is required for monitoring pressure in the pressure sensing endograft.

The endograft 18 is used in a system for monitoring pressures related to the performance of the implanted endograft itself, and for monitoring arterial pressure in the blood vessel.

In the aneurysm sac pressure embodiment, one or more pressure sensors S1 are embedded in the endograft 18 so that endograft material totally surrounds the sensors, with their pressure-sensing surfaces facing outward toward the aneurysm sac.

In a vascular pressure embodiment, one or more pressure sensors S2 are embedded in the endograft 18 so that endograft material totally surrounds the sensors, with their pressure-sensing surfaces facing inward toward the inside of the lumen to measure the patient's blood pressure.

In a hoop stress embodiment, one or more pressure sensors S3 are embedded near the ends of the endograft 18 so that endograft material totally surrounds the sensors, with their pressure-sensing surfaces facing outward toward the aortic wall. These sensors sense the clamping pressure caused by hoop stresses in the aortic wall that push the aortic wall against the sealing surface of the endograft under the action of the patient's blood pressure.

In a method embodiment, the signals generated by the sensors S1, S2, and S3 may be monitored, combined, and processed to provide pressure information to assist in the surgical installation of the endograft and also in monitoring the long-term performance of the endograft.

In any of the above embodiments, the electromagnetic energy can be wirelessly passed through the wall of the artery to supply power to the pressure sensors, and return signals from the pressure sensors may be remotely detected.

One advantage of the pressure-sensing endograft is that blood pressure within an aneurysm sac and the endograft lumen can be directly monitored.

A further advantage of the endograft is that the number of costly CT scans for patients with endovascular grafts can be reduced.

A further advantage of the endograft is that the blood pressure within an the aneurysm sac and within the endograft lumen can be periodically monitored using pressure differential trends for a more timely diagnosis of endoleakage.

A further advantage of the endograft is that the clamping pressure between the endograft and aorta can be monitored during the insertion procedure to determine when a secure fit has been achieved.

A further advantage of the endograft is that the frequency content and pulsatility of the dynamic pressure signals from the sensors measuring aneurysm sac pressure can be compared with the endograft luminal pressure to provide further indication of the performance of the endograft.

Yet a further advantage of the endograft is that the clamping pressure between the endograft and aorta can be monitored after the implantation procedure to monitor the integrity of the fit.

Yet a further advantage is that these pressures can be monitored wirelessly, so that no wires need penetrate the skin or the artery, and the associated complications of infection and thrombus generation can be avoided.

The pressure sensors Si through S3 illustrated in FIG. 1 are preferably embedded inside the endograft material, e.g. woven or knitted synthetic fiber such as Dacron, so that endograft material totally surrounds the sensor beneath a flattened portion to form a flat pressure-sensing surface. The endograft material smoothly blends back to the otherwise curved portion of the endograft.

These implanted pressure sensors measure absolute pressures. For clinical relevance, an external barometric reference sensor 30 in the monitoring system converts these pressures to gauge values.

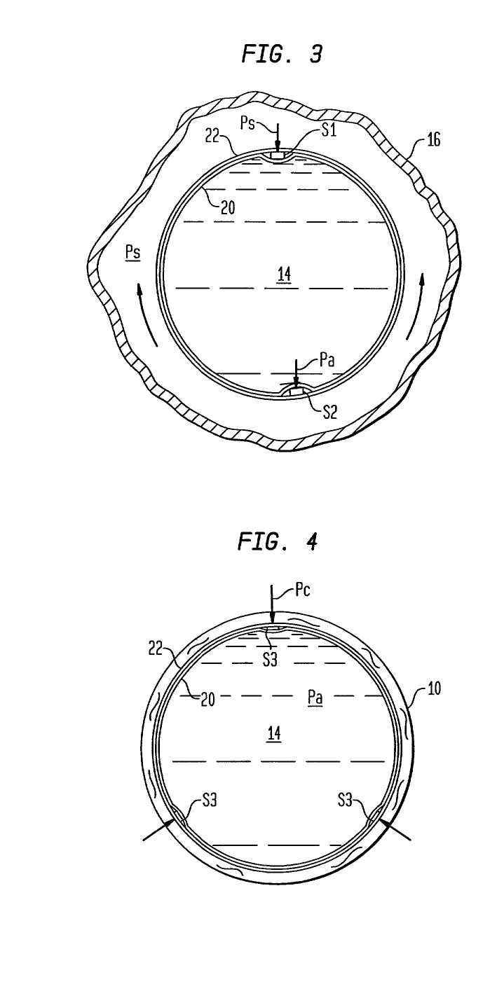

As illustrated in FIGS. 1 and 3, the first and second pressure sensors S1 and S2 are preferably located centrally in the middle of the endograft generally equally between the opposite ends of the sleeve. The flat diaphragm 26 of the first pressure sensor Si faces radially outwardly and contacts the outer sleeve layer 22 as illustrated in FIG. 3 for sensing external pressure Ps outside the endograft sleeve and within the aneurysm sac 16.

The first pressure sensor Si is therefore oriented for measuring pressure in the aneurysm sac 16. Multiple sensors S1 may be used for redundancy. The pressure-sensing surface 26 of sensor S1 faces outwardly toward the aneurysm sac. The pressure inside the aneurysm sac is communicated to the pressure sensor through the flattened endograft wall.

Correspondingly, the second pressure sensor S2 illustrated in FIGS. 1 and 3 has its flat diaphragm 26 facing radially inward and contacts the inner layer 20 of the endograft sleeve for sensing internal pressure Pa inside the endograft. The second pressure sensor S2 is preferably located centrally between the opposite ends of the endograft in the same plane as the first pressure sensor S1, but may be located at any axial location along the endograft where it measures the internal pressure of the flood flow therein.

The second pressure sensor S2 is therefore oriented for measuring pressure inside the endograft, which corresponds to the local vascular pressure within the lumen of the endograft. Multiple sensors S2 may be used for redundancy and to detect high flow resistances in the endograft.

The pressure-sensing surface 26 of sensor S2 faces inwardly toward the interior of the endograft. The outer layer 22 of endograft material pushes the pressure sensor inward sufficiently to flatten the inner layer 20 of the endograft material against the pressure-sensing surface. The pressure inside the endograft is communicated to the pressure sensor through this flattened endograft wall.

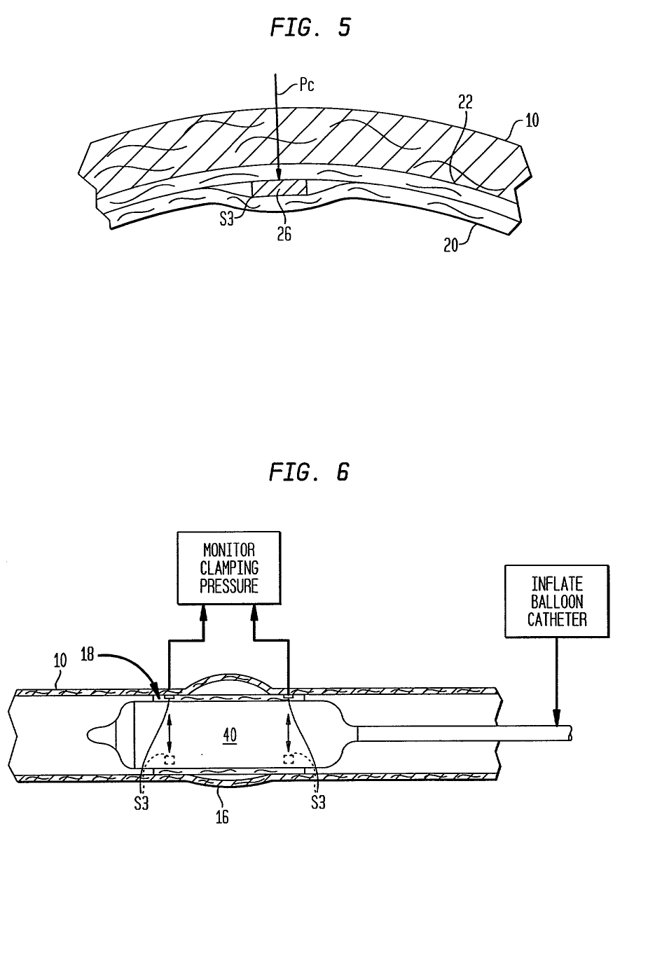

Illustrated in FIGS. 1, 4, and 5 are the third pressure sensors S3 preferably disposed in at least a single pair respectively located adjacent opposite ends of the endograft sleeve. Preferably, each end of the endograft includes three equiangularly spaced apart third pressure sensors S3 at a 120° spacing.

The end sensors S3 face radially outwardly as illustrated in FIGS. 4, 5, and 6 to measure contact or clamping pressure Pc exerted against the endograft after it is expanded against the inner wall of the vessel 10.

The third pressure sensors S3 are therefore provided for measuring the clamping pressure between the endograft and the aortic wall. The sensors S3 are outward facing and positioned adjacent to each end of the endograft where the endograft engages with the aortic wall. Multiple sensors S3 are preferably used at each end for redundancy, and to detect circumferential variations in the clamping pressure.

An endograft that is properly engaged with an aortic wall will expand the aortic wall elastically. A circumferential hoop stress will be established in the aortic wall that will tend to cause an even clamping pressure to be detected by the sensors S3.

Without gross non-uniformities in the aortic wall, this even distribution of the force through the hoop stress enables a single or small number of S3 pressure measurements to indicate the existence of good clamping pressure and a good seal around the entire circumferential sealing surface.

In contrast, an endograft that is not properly engaged with the aortic wall will not establish such a hoop stress and will cause a lower or nonexistent clamping pressure to be detected by the third sensors S3. This lower clamping pressure can be detected even if there is physical contact between the endograft and aorta. Thus the pressure sensors provide early warning of marginal clamping pressure and imminent leakage before a gross failure associated with loss of contact becomes visible through CT scans.

In the preferred embodiment illustrated in FIG. 1 the endograft includes all three types of pressure sensors S1, S2, and S3. The first pressure sensor S1 has its diaphragm 26 facing outwardly for detecting pressure Ps in the aneurysm sac 16. The second pressure sensor S2 has its diaphragm facing radially inwardly for detecting pressure Pa inside the endograft lumen. And, the third pressure sensors S3 are arranged in groups of three at opposite ends of the endograft with their diaphragms 26 facing radially outwardly for detecting the clamping pressure Pc.

For cardiovascular applications, it is important that the pressure sensor be securely fixed to its mounting to prevent undesirable liberation. FIG. 2 shows the pressure sensor mounted integral within layers of endograft material. The layers of endograft material surround the pressure sensor to prevent detachment.

The endograft material is typically a woven fabric. The weave of the endograft material should be sufficiently fine that the pores are substantially smaller than the pressure sensor notwithstanding any stretching or flexing of the endograft. This configuration minimizes the possibility of the sensor passing through a pore or otherwise becoming detached from the endograft.

The implanted endograft material produces a smooth, biocompatible tissue-incorporation that becomes an integral part of the sensor diaphragm. The pressure sensor diaphragm, such as silicon, is made to be stiffer than any tissue that may build up on its surface. Any thickening caused by further tissue buildup has a relatively small effect on the total sensor diaphragm stiffness and sensitivity.

In the preferred embodiment illustrated in FIG. 1, the endograft also includes an expandable stent 32 disposed coaxially with the two-ply sleeve thereof for structurally supporting the endograft when implanted in the blood vessel. The stent preferably surrounds the endograft sleeve and may be sewn to the fabric thereof. The inside of the endograft sleeve remains smooth for maintaining a substantially smooth continuous flowpath for the blood flowing therethrough during operation.

The stent generally has a single layer of expandable meshwork that undergoes plastic deformation to expand to form a rigid scaffolding to hold open the endograft in an artery. The stent is typically made of a biocompatible metal, such as Nitinol, stainless steel, or titanium.

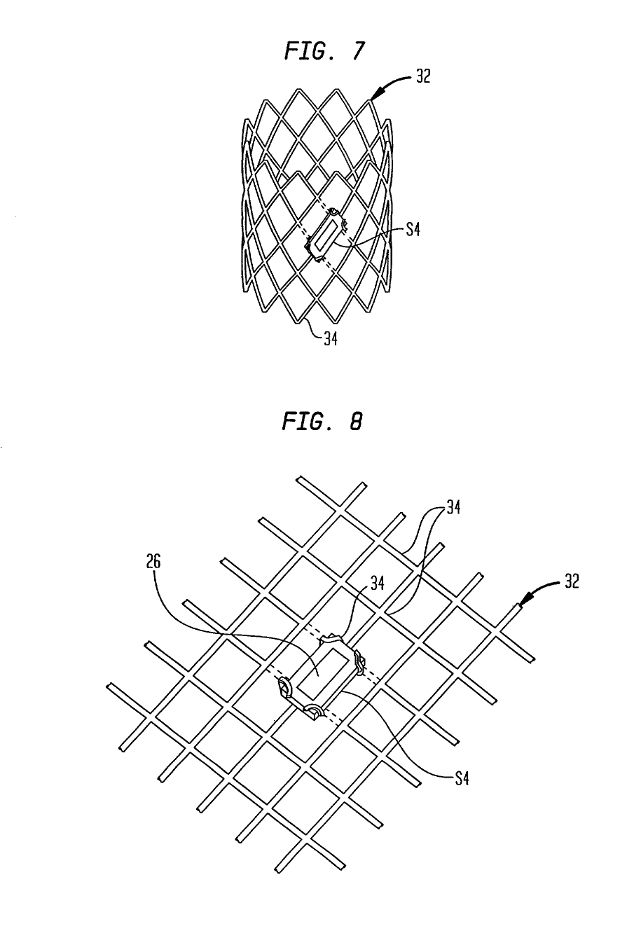

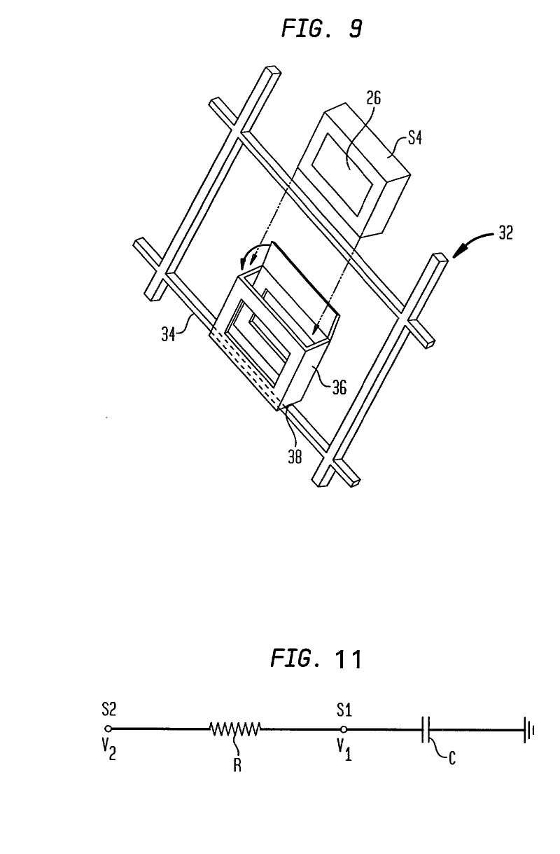

Illustrated in FIGS. 7 and 8 is the stent 32 of the endograft illustrated in FIG. 1 removed therefrom which may include another or fourth pressure sensor S4 fixedly joined to the stent. The stent includes a mesh or grid of interconnected wires 34, and the stent pressure sensor S4 is locally joined to at least one of the wires for retention thereto. The diaphragm 26 of the fourth pressure sensor S4 may face outwardly or inwardly as desired.

In the exemplary embodiment illustrated in FIG. 8, a plurality of the mesh wires 34 are cut and bent to mechanically trap the pressure sensor to the adjoining mesh wires. The bent mesh wires preferably trap the perimeter of the pressure sensor without covering the diaphragm 26 or preventing pressure sensing operation thereof.

Since the pressure sensor is supported by the cut mesh wires, it is freely carried along with the underlying uncut wires as the stent mesh is expanded in use to increase the cylindrical diameter of the tubular stent from its initially small-diameter collapsed form. The stent may therefore freely expand without local distortion around the retained pressure sensor.

FIG. 9 illustrates an alternate embodiment of the stent 32 in which the mesh wires 34 are not cut but define suitably sized openings or cells between the wires in which the pressure sensor may be mounted. In this configuration, a perforate frame or box 36 is fixedly joined to one or more of the mesh wires by a weld joint 38. The box may have six sides and a closure flap which is initially open for permitting the pressure sensor S4 to be inserted therein during assembly. The flap may then be simply bent closed for retaining the pressure sensor in the box.

The box preferably has two large windows on opposite sides thereof for permitting unobstructed access of the blood to the sensing diaphragm 26. Since the box is secured in one of the mesh cells, the stent may be readily expanded during implantation without restraint by the mounting box itself.

The endograft illustrated in the preferred embodiment in FIG. 1 incorporates integral pressure sensing capability which may be used to advantage during its initial implantation in the patient as well as for subsequent monitoring of endograft performance thereafter. For example, the endograft may be used for detecting leakage around the endograft after its implantation.

As illustrated in FIG. 1, the implanted endograft bridges the aneurysm sac 16, with opposite ends of the endograft contacting inner surfaces of the aorta 10 at opposite ends of the aneurysm to provide effective seals thereat and channel blood through the endograft instead of the aneurysm.

The first pressure sensor S1 may then be used to detect external pressure outside the implanted endograft and inside the aneurysm sac for detecting pressure of any blood leakage therein. The second pressure sensor S2 may be correspondingly used to detect internal pressure inside the endograft due to the pressure of the blood 14 channeled therethrough.

By simply comparing the external and internal pressures detected by the first and second pressure sensors, an indication of endoleakage may be derived. The external pressure of the endograft should be substantially lower than the internal pressure for normal, sealed operation of the opposite endograft ends.

FIG. 6 illustrates an exemplary method of using or implanting the endograft 18. The endograft 18 in initially collapsed form is mounted around a conventional balloon catheter 40 and conventionally guided through a suitable artery to a desired position inside the aorta 14 to internally bridge the aneurysm sac 16. The balloon catheter may then be expanded for in turn expanding the endograft and its supporting stent into engagement with the inner surface of the aorta.

The two groups of third pressure sensors S3 may then be used for monitoring the clamping pressure of engagement of the endograft with the aorta wall at opposite ends of the endograft sleeve. Endograft expansion by the balloon catheter may be terminated upon reaching a suitable value of monitored clamping pressure as detected by the third pressure sensors.

Because the sensors are embedded in the endograft, separate surgical procedures for implantation thereof are not necessary. Furthermore, because the embedded pressure sensors are read by telemetry, no separate surgical procedure is required for monitoring the pressures needed to diagnose the pressure integrity of the endograft. Thus, follow-up diagnostics for pressure integrity using these sensors are a non-invasive procedure.

The patient's blood pressure may vary considerably from moment to moment. These pressure variations may or may not be related to the integrity of the endograft or any leakage of blood into the abdomen. These blood pressure variations may result in artifacts generated in the pressure of the aneurysm sac.

Thus, to reduce these artifacts, the differential pressures between the endograft luminal pressure and the aneurysm sac pressure may be monitored. Additionally, these pressure differences may be further analyzed in terms of mean pressure, pulse pressure, and frequency content.

The following example illustrates one possible analysis approach for processing the pressure sensor signals:

Pa=aortic pressure in the endograft lumen 18

Ma=mean pressure in the endograft lumen

Ppa=pulsatile pressure in the endograft lumen

Pa=Ma+Ppa

Ps=pressure in the aneurysm sac

Ms=mean pressure in the aneurysm sac

Pps=pulsatile pressure in the aneurysm sac

Ps=Ms+Pps

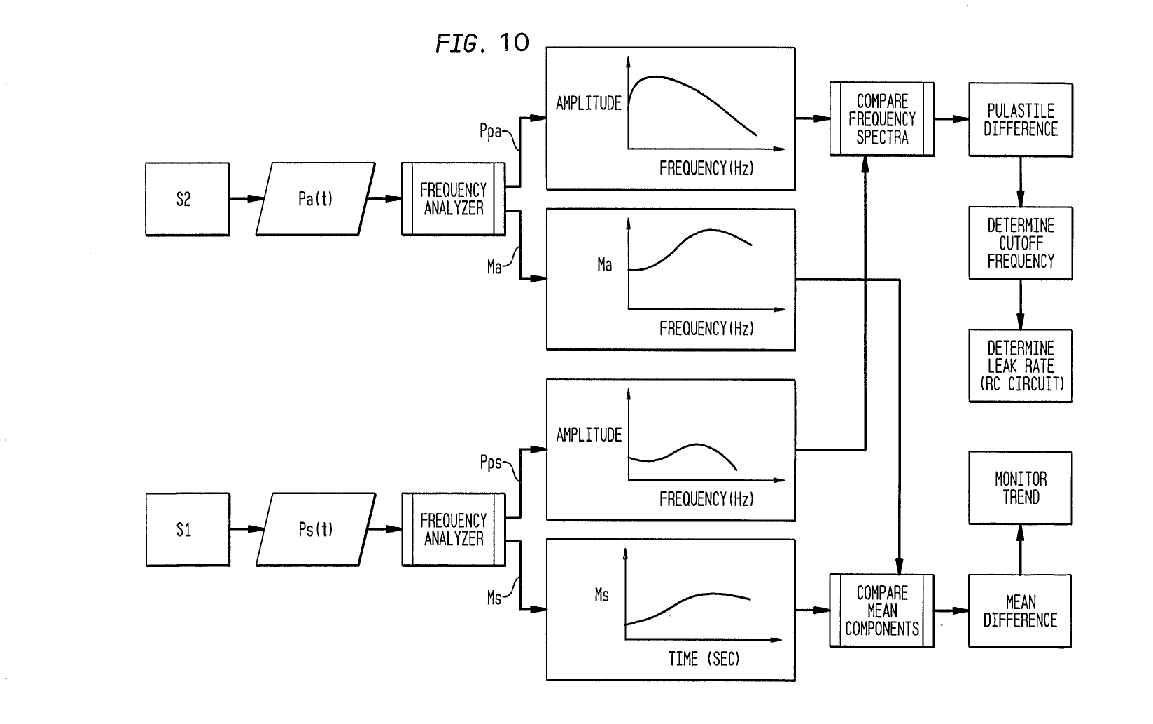

A schematic representation of these parameters as a function of time (t) is illustrated in FIG. 10.

The monitored pressure differences are shown below:

Pulsatile difference=Ppa−Pps

Mean difference=Ma−Ms

For example, at the time of the endograft insertion, the mean difference may be zero, but there can be an immediate and significant pulsatile difference as soon as the aneurysm is isolated. Over time, the mean difference should increase as the blood in the aneurysm sac transforms into a shrunken thrombus. These distinctions in mean difference and pulsatile difference may further help eliminate other artifacts. For example, abdominal intestinal bloat that may decrease the mean difference but not significantly change the pulsatile difference.

If an endoleak is present, the amount of endoleak can be inferred from the following approach. An analysis of the attenuation of the frequency content of pressure signals reported by S1 and S2 can be an indicator of the impedance of the leak path and the degree to which a high resistance, tight seal has been achieved. This impedance can be analyzed using techniques well known in the art of circuit design.

In the equivalent circuit of FIG. 11, a resistor-capacitor circuit models the performance of the pressure sensing system. In FIG. 11:

R=the resistance to flow from the artery to the aneurysm sac, in mmHg/(mL/second)

C=the capacity of the aneurysm sac, in mL/mmHg

Pps=the pulsatile pressure in the aneurysm sac reported by sensor S1 and corresponds to the voltage V1 in an equivalent electrical circuit

Ppa=the pulsatile pressure in the artery reported by sensor S2, and corresponds to the voltage V2 in an equivalent electrical circuit

Flow=(Ppa−Pps)/R, the flow into the sac in mL/second, and corresponds to current flow in the equivalent electrical circuit

T=R×C, the characteristic fill time of the sac, in seconds, and corresponds to the characteristic saturation time of the equivalent electrical circuit.

Where the period of the frequency component is small compared to the characteristic time T, i.e., a high frequency component, a small fraction of the pulsatile arterial pressure Ppa is transmitted to the aneurysm sac as Pps.

Where the period of the frequency component is large compared to the characteristic time T, i.e., a low frequency component, most of pulsatile arterial pressure Ppa is transmitted to the aneurysm sac as Pps. The cutoff frequency of this low-pass filter (1/RC) can be used to infer the value of the resistance R with respect to the capacity C.

In the field of electrical circuits and signal processing, the characteristic time constant of an RC-circuit is approximately t=R*C (in seconds) and the characteristic cutoff frequency is approximately f=1/R*C (in Hz).

In the flow analogue to the electrical circuit, R becomes the resistance to flow, in the form of Pressure/Flow through the leak, in units of mmHg/(cc/sec). The capacitance C becomes the compliance of the aneurysm sac, in units of cc/mmHg. The time constant t=RC retains the units of seconds, and frequency f=1/RC retains the units of Hz. Monitoring this cutoff frequency f for changes enables the physician to also monitor changes in the product RC, an indication of leakage rate.

If an approximation is used for the compliance C of the aneurysm sac, the leakage resistance R can be directly calculated as R=1/fC. The capacitance Cis related to the size of the aneurysm sac, which can be seen through radiological images. A typical compliance for an expanded aneurysm sac can be in the range of 1 cc/mmHg. For a cutoff frequency of 5 Hz, the leakage resistance can be approximated by R=1/(5*Hz*1* cc/mmHg)=0.2 mmHg/(cc/sec). These calculations are very approximate and the changes in values should be followed rather than the absolute values.

As illustrated in FIG. 10, in a preferred method of using the implanted endograft, the mean components of the external pressure Ps(t) and internal pressure Pa(t) may be compared, in a suitable signal processor for example, for detecting endoleakage. Furthermore, a conventional frequency analyzer may be used to uncover the frequency spectra of the pulsatile components of the external and internal pressures as distinct from the mean components thereof for detecting endoleakage.

For example, attenuation of the pulsatile components and cutoff frequency therefrom may be determined for detecting the endoleakage in the form of the RC leakage rate described above.

Finally, one way to prevent the progression of further aneurysms is to monitor for hypertension, treat the hypertension with appropriate drugs, and monitor for drug effectiveness and patient compliance. Thus, the ability for the patient ambulatory monitoring of his/her blood pressure may be a valuable clinical tool.

The pressure sensing endograft described above integrates minute pressure sensors therein for improving both performance of the initial implantation thereof, as well as monitoring use of the endograft over time. Telemetry reading of embedded pressure sensors eliminates need for any surgical procedures in monitoring endograft performance. And, continual monitoring of endograft performance ensures its effectiveness in preventing leakage into the aneurysm.

While there have been described herein what are considered to be preferred and exemplary embodiments of the present invention, other modifications of the invention shall be apparent to those skilled in the art from the teachings herein, and it is, therefore, desired to be secured in the appended claims all such modifications as fall within the true spirit and scope of the invention.

Accordingly, What is desired to be secured by Letters Patent of the United States is the invention as defined and differentiated in the following claims in which we claim:

1. An endograft comprising:
a tubular sleeve having integral inner and outer layers;
a pressure sensor embedded between said layers and covered thereby; and
said sleeve being flexible at said pressure sensor to permit transfer of pressure through said sleeve to said pressure sensor.

2. An endograft according to claim 1 wherein said pressure sensor includes a flat pressure sensing diaphragm, and said sleeve is flexible to conform with said flat diaphragm.

3. An endograft according to claim 2 wherein said diaphragm faces radially outward and contacts said outer layer for sensing pressure external to said sleeve.

4. An endograft according to claim 2 wherein said diaphragm faces radially inward and contacts said inner layer for sensing pressure internal of said sleeve.

5. An endograft according to claim 2 wherein said pressure sensor is located centrally between opposite ends of said sleeve.

6. An endograft according to claim 2 further comprising:
a first pressure sensor having said diaphragm thereof facing radially outward and contacting said outer layer for sensing pressure external to said sleeve; and
a second pressure sensor having said diaphragm thereof facing radially inward and contacting said inner layer for sensing pressure internal of said sleeve.

7. An endograft according to claim 2 further comprising a pair of said pressure sensors located adjacent opposite ends of said sleeve.

8. An endograft according to claim 7 further comprising three equiangularly spaced apart pressure sensors located adjacent each of said sleeve opposite ends and facing radially outwardly.

9. An endograft according to claim 2 further comprising:
a first pressure sensor having said diaphragm thereof facing radially outward and contacting said outer layer for sensing pressure external to said sleeve;
a second pressure sensor having said diaphragm thereof facing radially inward and contacting said inner layer for sensing pressure internal of said sleeve; and
two pairs of three third pressure sensors equiangularly spaced apart adjacent opposite ends of said sleeve and facing radially outwardly.

10. An endograft according to claim 2 wherein said sleeve layers are fabric.

11. An endograft according to claim 2 wherein said inner and outer layers are coextensive between opposite ends of said sleeve.

12. An endograft according to claim 2 wherein said pressure sensor further includes an inductor for telemetric detection of pressure sensed thereby.

13. An endograft according to claim 2 further comprising a stent disposed coaxially with said sleeve for support thereof.

14. An endograft according to claim 13 further comprising another pressure sensor fixedly joined to said stent.

15. An endograft according to claim 14 wherein said stent includes a mesh of interconnected wires, and said stent pressure sensor is locally joined to at least one of said wires.

16. An endograft according to claim 15 wherein a plurality of said wires are cut and bent to trap said stent pressure sensor to uncut ones of said wires.

17. An endograft according to claim 15 further comprising a perforate box fixedly joined to said one stent wire, and said stent pressure sensor is trapped inside said box.

18. A method for using said endograft according to claim 6 to detect leakage therearound comprising:
implanting said endograft inside a body vessel having an aneurysm sac, with opposite ends of said endograft contacting inner surfaces of said vessel at opposite ends of said sac;
using said first pressure sensor to detect external pressure outside said endograft and inside said sac;
using said second pressure sensor to detect internal pressure inside said endograft; and
comparing said external and internal pressures to detect leakage.

19. A method according to claim 18 further comprising comparing mean components of said external and internal pressures to detect said leakage.

20. A method according to claim 18 further comprising comparing frequency spectra corresponding with pulsatile components of said external and internal pressures to detect said leakage.

21. A method according to claim 19 further comprising determining attenuation of said pulsatile components and cutoff frequency therefrom to detect said leakage.

22. A method of implanting said endograft according to claim 8 comprising:
expanding said endograft using a balloon catheter inside a body vessel to bridge an aneurysm sac therein;
monitoring clamping pressure of engagement of said endograft with said vessel using said pressure sensors at opposite ends of said sleeve; and
terminating said endograft expansion at a suitable value of monitored clamping pressure.
